(19) United States
(12) Patent Application Publication
(10) Pub. No.: US 2013/0096550 A1
(43) Pub. Date: Apr. 18, 2013

(54) ABLATIVE CATHETER WITH ELECTRODE COOLING AND RELATED METHODS OF USE

(71) Applicant: Boston Scientific Scimed, Inc., Maple Grove, MN (US)

(72) Inventor: DAVID M. HILL, ORONO, MN (US)

(73) Assignee: BOSTON SCIENTIFIC SCIMED, INC., MAPLE GROVE, MN (US)

(21) Appl. No.: 13/654,250

(22) Filed: Oct. 17, 2012

Related U.S. Application Data
(60) Provisional application No. 61/548,608, filed on Oct. 18, 2011.

(51) Int. Cl. A61B 18/14; A61B 18/18

(52) U.S. Cl. 606/33; 606/41

(57) ABSTRACT

Medical devices and methods for making and using medical devices are disclosed. An example medical device may include an ablative catheter system including an elongate member having a proximal end, a distal end, and a lumen extending there between. An end-effector may be disposed at the distal end of the elongate member. The end-effector may include an expandable frame. A membrane may be supported on the frame. The membrane may be configured to partially occlude fluid flow upon frame expansion. One or more electrodes may be placed on the end-effector. The system may also include a control member that is configured to shift the end-effector between a collapsed state and the frame expansion state.

### CROSS-REFERENCE TO RELATED APPLICATIONS

This application claims priority under 35 U.S.C. §119 to U.S. Provisional Application Ser. No. 61/548,608, filed Oct. 18, 2011, the entirety of which is incorporated herein by reference.

### FIELD OF THE INVENTION

Embodiments of the present disclosure relate generally to medical devices suitable for use in body tissue modulation and ablation. In particular, embodiments of the instant disclosure relate to structures and methods for cooling medical devices employed in tissue modulation and ablation.

### BACKGROUND OF THE INVENTION

Radio frequency ablation (RFA) is a relatively new medical procedure in which tissue is ablated using the heat generated from the radio frequency waves. Unlike the conventional procedures, the RF current does not interfere with the nervous or cardiac system and can be used as a minimally invasive procedure without a general anesthetic. Further, RFA procedures generally require image guidance, such as X-ray screening, CT scan, or ultrasound.

Many nerves, such as renal nerves, run in close proximity to blood vessels and thus can be accessed intravascularly through the blood vessel walls. This treatment, however, may result in thermal injury to the artery walls. The treatment can also produce undesirable side effects, such as blood damage and clotting, and the high temperature can produce protein fouling of the electrode. One of the ways to reduce thermal damage involves cooling the nerve ablation region by natural blood flow through the renal artery. An alternate approach for reducing artery wall damage involves placing the RF electrodes a short distance away from the artery wall. Though effective, that process requires precise spacing of electrodes from the artery wall, which is difficult to achieve.

Therefore, there exists a need for a system which reduces vessel wall damage by cooling the RF electrodes and places them automatically at a controlled distance from the vessel wall.

### SUMMARY OF THE INVENTION

One embodiment pertains to an ablative catheter system comprising an elongate member having a proximal end and a distal end. The elongate member may be a catheter and include one or more lumens extending along its length. An end-effector may be disposed at the distal end of the elongate member, the end-effector including an expandable frame and a membrane supported on the frame. The membrane may be configured to partially occlude fluid flow upon frame expansion. One or more electrodes may be placed on the end-effector and are configured to ablate or otherwise modulate tissue. The system also includes a control member configured to translate the end-effector between a collapsed state and the frame expansion state. Such a control member may be a sheath, a pull wire or the like.

Upon expansion, the membrane increases radially in size from a proximal end and may have a generally conical shape and may further include pleats or other concavities. In some embodiments, the membrane is non-pourous or is otherwise generally impervious to blood flow. The membrane may be impermeable to radio-frequency energy. The membrane may extend distally from the distal end of the elongate member or may be spaced longitudinally from the distal end of the elongate member.

The one or more electrodes are placed on an outer surface of the membrane, an inner surface of the membrane, on the expandable frame, a separate electrode-carrying structure or other suitable location. The electrodes are preferably placed so that, upon expansion of the end-effector in a vessel having walls, the one or more electrodes are spaced apart from the vessel wall. The expandable frame may include a plurality of struts. The plurality of struts may extend from the distal end of the elongate member to a distal end of the membrane. The plurality of struts may extend from the distal end of the elongate member distally past a distal end of the membrane. Upon expansion, the plurality of struts and the membrane form a generally conical shape. The distal ends of the struts may include distal ends that are turned inwardly or have some other atraumatic feature.

In some contemplated embodiments, the expandable frame may include a first section and a second section distal the first section, and wherein the expandable frame has an expanded configuration wherein the first section increases radially in size distally and the second section decreases radially in size distally (and so form a double-cone or football-shaped frame). The expandable frame comprises longitudinally struts that converge to a distal end of the second section. The membrane may be disposed proximal the second section or may be disposed on the first and second sections. The expandable frame comprises an atraumatic distal end.

The control member may be coupled to the proximal end of the end-effector and may extend proximally within the lumen of the elongate member. Alternatively, the control member may be a tube or sheath that is slidable over the end-effector to move the end-effector between a closed position and an open position or to allow an end-effector that is biased in an open position to assume the open position upon withdrawal of the sheath.

Some embodiments pertain to a method of performing an intravascular procedure, comprising the steps of providing a system comprising elongate member having a distal region including an expandable end-effector having a blood impermeable membrane and an electrode, positioning the end-effector intravascularly at a region of interest, expanding the membrane to partially occlude blood flow and form pleats in the membrane, and activating the electrode. The membrane may have a proximal end and a distal end such that the membrane has a perimeter at the distal end that is larger than the perimeter at the proximal end. A system used in the method may be any of the systems described herein.

An example medical device for modulating nerve activity may include a sheath. An elongate shaft may be disposed in the sheath. The shaft may have a distal end. An expandable frame may be attached to the shaft. The frame may include a plurality of struts and a membrane attached to the struts. The frame may be configured to shift between an expanded configuration and a collapsed configuration. The membrane may be configured to partially occlude blood flow through a blood vessel when the frame is in the expanded configuration. One or more electrodes coupled to the frame.

Another example medical device for modulation of renal nerve activity may include a sheath. An elongate shaft may be slidably disposed within the sheath. The shaft may have a distal region. A self-expanding umbrella frame may be attached to the shaft. The frame may include a plurality of struts and a membrane attached to the struts. The frame may be configured to shift between a collapsed configuration when the frame is disposed within the sheath and a conical configuration when the sheath is disposed proximally of the frame. One or more electrodes may be coupled to the frame. The membrane may define a plurality of pleated regions that extend radially inward relative to the struts when the frame is in the conical configuration. The pleated regions may be configured to increase blood flow adjacent to the electrodes.

Method for modulating renal nerves are also disclosed. An example method may include providing a renal nerve modulation device. The renal nerve modulation device may include a sheath. An elongate shaft may be slidably disposed within the sheath. The shaft may have a distal region. A self-expanding umbrella frame may be attached to the shaft. The frame may include a plurality of struts and a membrane attached to the struts. The frame may be configured to shift between a collapsed configuration when the frame is disposed within the sheath and a conical configuration when the sheath is disposed proximally of the frame. One or more electrodes may be coupled to the frame. The membrane may define a plurality of pleated regions that extend radially inward relative to the struts when the frame is in the conical configuration. The pleated regions may be configured to increase blood flow adjacent to the electrodes. The method may also include advancing the renal nerve modulation device through a blood vessel to a position within a renal artery and proximally retracting the sheath relative to the frame. Proximally retracting the sheath relative to the frame may shift the frame from the collapsed configuration to the conical configuration. The method may also include activating at least one of the one or more electrodes.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The accompanying drawings, which are incorporated in and constitute a part of this specification, illustrate embodiments of the invention and together with the description, serve to explain the principles of the invention.

FIGS. 1A and 1B illustrate an exemplary embodiment of the distal end of a renal nerve ablation system according to an embodiment of the present disclosure.

FIG. 2 depicts an alternate embodiment of the distal end of the renal nerve ablation system shown in FIG. 1.

FIGS. 3A and 3B illustrate an exemplary distal end of a renal nerve ablation system, shown in FIG. 1A with an inner expansion member.

FIGS. 4A and 4B depict a further alternate embodiment of the renal nerve ablation system shown in FIG. 1, shown in expanded and collapsed states, respectively.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Certain treatments require the temporary or permanent interruption or modification of select nerve function. One example treatment is renal nerve ablation, which is sometimes used to treat conditions related to hypertension and/or congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

While the devices and methods described herein are discussed relative to renal nerve modulation, it is contemplated that the devices and methods may be used in other treatment locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, cardiac ablation, pulmonary vein isolation, tumor ablation, benign prostatic hyperplasia therapy, nerve excitation or blocking or ablation, modulation of muscle activity, hyperthermia or other warming of tissues, etc.

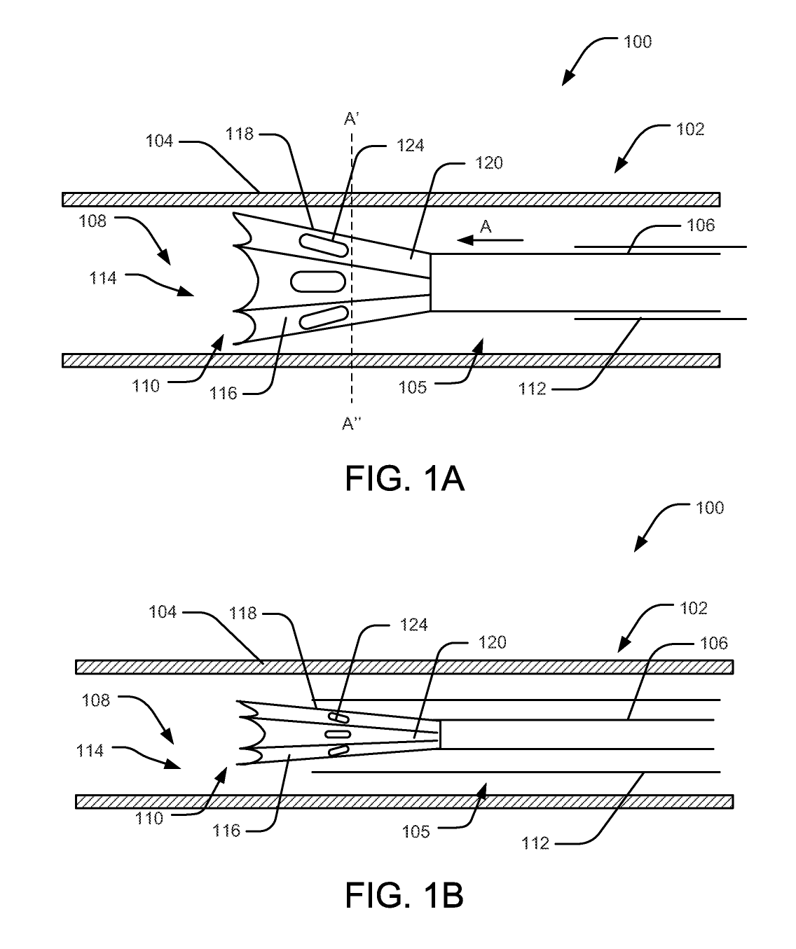

FIGS. 1A and 1B illustrate an exemplary embodiment of a portion of a renal nerve ablation system 100 disposed within a body lumen or blood vessel 102, such as a renal artery. Both figures depict an ablation catheter 105, with FIG. 1A depicting the ablation catheter 105 in a deployed or expanded state and FIG. 1B depicting ablation catheter 105 in a partially collapsed state. The catheter 105 includes an elongate shaft or member 106 having a distal end region 108 and a proximal end (not shown). The elongate member 106 can vary in form and may be similar to conventional medical devices including catheters, guidewires, endoscopic devices, and the like. For example, the elongate member 106 may include one or more lumens, such as a guidewire lumen or one or more inflation lumens. The lumens may be configured according to the needs of a particular intervention. Alternatively, the elongate member 106 may take the form of a solid shaft or wire.

An end-effector 114 may be disposed at the distal end of the elongate member 106. Generally, end-effector 114 is configured to shift between an expanded form or configuration and a collapsed form or configuration (suitable for being maneuvered through body lumens within the body lumen 102). In at least some embodiments, end-effector 114 may be configured to shift between the expanded configuration and the collapsed configuration by shifting the position of the elongate member 106 relative to a sheath 112. For example, elongate member 106 may be positioned so that the end-effector 114 is positioned distally of the sheath 112. When so positioned, the end-effector 114 may shift to the expanded configuration (e.g., as shown in FIG. 1A). This may be due to end-effector 114 including super elastic and/or shape memory materials such as a nickel-titanium alloy. In other words, end-effector 114 may be “self-expanding”. When in the expanded configuration, the end-effector 114 may partially occlude the flow of blood through blood vessel 102. When the elongate member 106 is shifted proximally relative to the sheath 112, the sheath 112 may cause the end-effector 114 to shift toward the collapsed configuration (e.g., FIG. 1B illustrates the elongate member 106 partially shifted relative to the sheath 112 such that the sheath 112 engages the end-effector 114 and begins to collapse the end-effector 114). It can be appreciated that the elongate member 106 may be further shifted proximally relative to the sheath 112 such that the end-effector 114 is fully collapsed and contained within the sheath 112. It can also be appreciated that shifting the end-effector 114 between the expanded configuration and the collapsed configuration may include movement of the elongate member 106, the sheath 112, or both. Other expansion mechanisms are also contemplated. Some of these alternative expansion mechanisms are disclosed herein.

When in the expanded configuration, end-effector 114 may be shaped like a cone or otherwise have a generally conical shape. End-effector 114 partially occludes the interior of vessel 102 either by having a distal end that is smaller than the cross-section of the body lumen 102 and/or by including pleats or folds.

End-effector 114 may include a frame (indicated generally at 120) that includes a plurality of struts 118 and a membrane 116 lying over and secured to the struts 118. Each strut 118 may extend from the distal end of the elongate member 106, where each strut 118 is fixed. The number and length of the struts 118 depends upon the particular application, as will be understood by those in the art. In the embodiment illustrated, six struts 118 are employed, each strut being about 1-5 cm long. Variations are contemplated, however, that include any suitable number of struts 118 including one, two, three, four, five, six, seven, eight, nine, ten, or more struts 118.

The end-effector 114 may be equipped with a control member that may urge each strut 118 to the expanded state shown in FIG. 1A, where the frame 120 is shown in an expanded state. As explained below, it will be advantageous in many situations to maintain the frame 120 in a collapsed state, as shown in FIG. 1B. The control member can take a number of forms. For example, as indicated above each strut 118 can be biased to an expanded state (e.g., the end-effector 114 and/or frame 120 may be “self-expanding”), with expansion of the frame 120 caused by the withdrawal of a sheath 112 from a restraining position over the frame 120. In other words, the self-expanding nature of the frame 120 may render the frame 120 itself a “control member”. Alternatively, each strut 118 can be fitted with a spring that urges the strut 118 angularly away from the longitudinal axis of the elongate member 106. In other embodiments, an expandable ring, pull wire, central balloon or other suitable structure can be employed to expand the end-effector 114. These are just examples. Other expansion mechanisms are contemplated.

When freed from restraint, the control member may cause a portion of each strut 118 to move radially away from the longitudinal axis of elongated member 106, pushing membrane 116 outwardly. That expansion continues until the struts 118 are at their furthest expanded state or have encountered the wall 104 of the body vessel. When the struts 118 bear on walls 104, the membrane 116 can be described as forming a pleated structure, with the membrane portions lying between struts 118 assuming cupped or concave forms. In other words, the pleated regions of the membrane 116 may extend radially inward relative to the struts 118 and/or frame 120. Such a configuration allows the membrane to partially occlude the vessel while channeling the blood flow through the pleats. When doing so, the flow of blood may increase along the pleats and/or along electrodes 124 positioned generally along the frame 120. This may be desirable for a number of reasons. For example, the increased blood flow along the electrodes 124 may aid in dissipating heat that might be generated during activation of the electrodes 124.

As indicated above, one or more electrodes 124 may be provided along the frame 120 and may be located on the outside surface of membrane 116, on the inside surface of membrane 116, on the struts 118, on a separate electrode bearing structure or other suitable location. In at least some embodiments, electrodes 124 are RF (radio frequency) electrodes. Other electrodes are contemplated including laser electrodes, microwave electrodes, ultrasound transducers, or the like. Electrodes 124 may be sized and located to provide a desired RF field, capable of accomplishing the desired nerve ablation. In the illustrated embodiment, electrodes 124 are located between each pair of struts 118, spaced from the distal end of the membrane 116. In the illustrated configuration, electrodes may be formed of a metal electro-deposited or painted on the membrane. Furthermore, each electrode 124 is appropriately connected to an RF energy source (not shown). Alternative locations for electrodes may be the tips of struts 118, on the inner side of membrane 116, or any other suitable location. The electrodes 124 are illustrated as oblongs, but may be oval, circular, or other suitable shape.

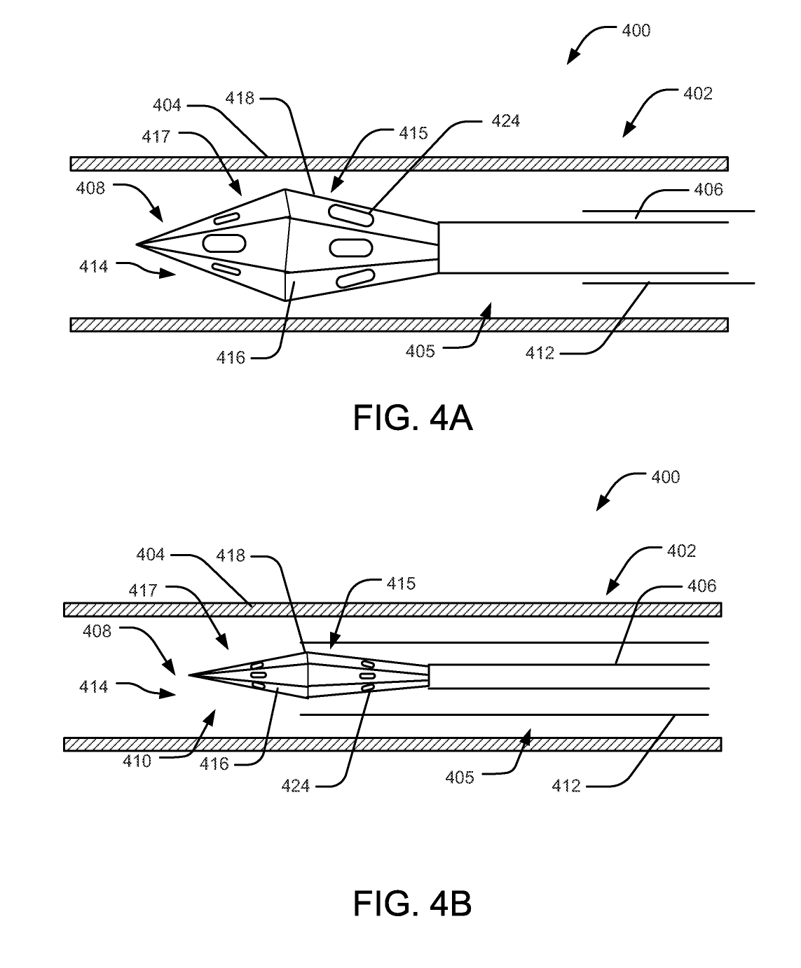

In alternative embodiments, electrodes 124 can be attached to other structures, rather than directly on the frame 120. For example, an electrode 124 can be suspended by a separate support strut, or between two struts. Additionally, the frame 120 may be configured as a double cone (e.g., as shown in FIGS. 4A-4B), with struts 118 extending further distally to taper in and join together at a distal end (not shown). Such extended or additional struts can provide improved passage of the cone within the vessel, as explained in detail below. The additional struts may be bare or may be partially covered by occlusive material. In some embodiments, multiple electrodes 124 may be used simultaneously or in sequence for ablating multiple or circumferential locations without repositioning the elongate member 106.

Struts 118 can be formed of any material possessing requisite characteristics of resilience and stiffness. Suitable materials include nitinol or other shape-memory or highly elastic materials, or stainless steel, or other alloys, or elastic polymer, or combinations. Where struts 118 are designed to impinge upon walls 104, each strut 118 may have sufficient contact area to minimize mechanical trauma to the vessel wall. For example, each strut 118 may have a wall-contacting pad or may be curved inwardly at the distal end to form a convex atraumatic contact with the vessel wall 104.

The membrane 116 may be formed of a relatively thin, flexible material, such as polyester, fluoropolymer, or other polymers, flexible metallic structures, coatings, or combinations. As will be appreciated from considering the description of operation below, the material for membrane 116 can be selected to be impermeable to bodily fluids or to permit a desired amount of seepage or leakage. Materials such as non-porous versions of embolic protection filter membrane materials produce impermeable membranes. The membrane material can be attached to struts 118 by any suitable attachment means or method, such as adhesive, thermal bond, or the like.

When deployed within the body lumen 102, membrane 116 at least partially occludes the flow of blood or other fluid within the lumen. The degree of occlusion can be controlled by the extent to which the distal ends of struts 118 expand, by the shape of the membrane 116 between adjacent struts 118, and by the material of membrane 116. For example, struts 118 can be designed to expand completely within body lumen 102, impinging upon walls 104, or that expansion can be controlled to leave some degree of space between the distal tips of struts 118 and walls 104. Further, the shape of membrane 116 lying between adjacent struts 118 can be scalloped (depicted generally at reference number 110) to permit a desired amount of flow around the deployed frame 120, as shown in FIG. 1A. Other membrane shapes can be selected to provide desired amounts of flow past the end-effector 114. For example, apertures could be provided within membrane 116, increasing the amount of allowed flow. Finally, the permeability of membrane 116 can also help determine the amount of fluid flow past end-effector 114.

An effect of reducing the cross-sectional area of lumen 102 in a relatively localized area is an increase in the flow velocity within the lumen at that localized area. This increased velocity concomitantly increases the cooling effect of the fluid. Because the remaining flow is almost totally confined to the portion of the lumen adjacent to the walls 104, the increased cooling capacity of the fluid results in improved heat removal from the vessel walls 104. In that manner, ablation effectiveness may be improved while minimizing danger to surrounding tissue. For example, the fluid flow past the frame 120 illustrated in FIG. 1A will be increased and this higher velocity fluid flow will be in contact with the vessel wall, resulting in improved cooling in that location.

FIG. 1B illustrates frame 120 in a partially collapsed state, carried within a sheath 112. Working with the cone in its collapsed state allows operators to maneuver an endoscopic device carrying elongate member 106 through bodily lumens to a desired surgical site. When located at the site, conventional pull-or push-wires can be employed to extend end-effector 114 from the sheath 112 or a sheath may be withdrawn to allow an end-effector 114 to expand.

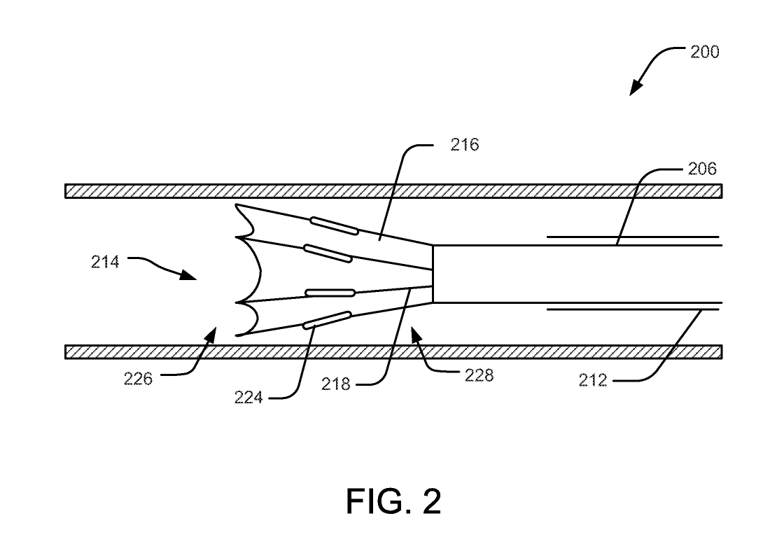

FIG. 2 depicts the distal end of a renal nerve ablation system 200 having an elongate member 206, with an end-effector/frame 214 at its distal end. The frame 214 may include one or more struts 218 having RF electrodes 224 coupled thereto for performing nerve ablation. The frame 214 may also include membrane 216. The components of the renal nerve ablation system 200 perform similar functions as described in relation with FIGS. 1A and 1B. As depicted, the frame 214 may include a first section 226 and a second section 228 proximal to the first section 226. Upon expansion, first section 226 may increase radially in size relative to the second section 228. The membrane 216 may be connected to the struts 218 at the second section 228 and expand radially with the second section 228. As depicted, sheath 212 may be slidable relative to elongate member 206, which may shift the frame 214 between a collapsed and an expanded state.

In this embodiment, RF electrodes to 224 are positioned on the struts 218 rather than on the surface of membrane 216. A number of alternative positions for location of the RF electrodes 224 are contemplated based on the delivery of a desired RF field at a target location.

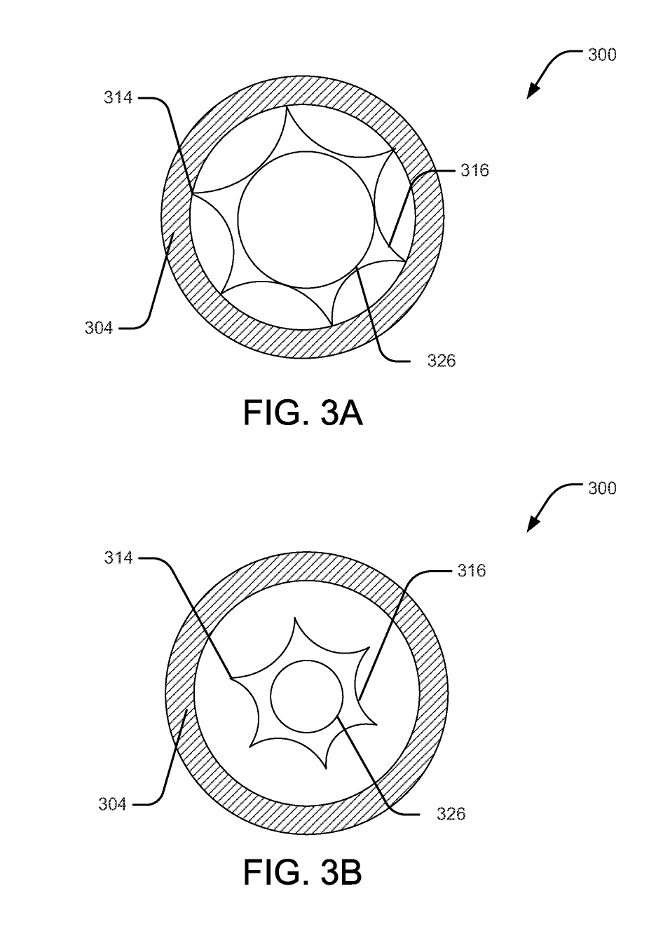

FIGS. 3A and 3B illustrate an alternative embodiment of a renal nerve ablation system 300 taken along the A-A′ plane (FIG. 1). FIG. 3A depicts a cross-sectional view of the frame 314 with inflated inner expansion member 326 and FIG. 3B depicts a collapsed cross-sectional view of the frame 314. As illustrated, the frame 314 includes an inner expansion member 326 disposed on the elongate member and a membrane 316 supported on the frame 314. The inner expansion member 326 may further occlude the blood vessel 304. The inner expansion member 326 may be inflated by injecting inflation fluid through a connected lumen (not shown) or may be an expandable structure. The inner expansion member 326 may have a circular cross-section, but it is contemplated that the inner expansion member 326 may have any desired shape or size. The inner expansion member 326 may or may not contact the elongate member 306 to vary the stiffness of the elongate member 306 for use in various vessel diameters. In one embodiment, the inner expansion member 326 may be an inflatable balloon. Alternatively, the inner expansion member 326 may be an expandable structure, for example, a stent-type structure.

As depicted, the inner expansion member 326 may be placed proximal of the A-A′ plane, such that the electrodes are spaced apart from both the inner inflatable member 326 and the vessel wall 304. During nerve ablation, the elongate member is advanced to the site of operation in a collapsed state. Once there, the operator may inflate the inner expansion member 326 to expand the frame 314 within the blood vessel and place the RF electrodes 324 some distance apart from the vessel wall 304. This off-wall positioning of electrodes along with increased blood velocity provided by the expanded frame 314 increases convective cooling of RF electrodes 324 and reduces vessel wall injury and blood damage.

FIGS. 4A and 4B depict a further alternate embodiment 400 of a renal nerve ablation catheter 405 disposed within blood vessel 402. This system is similar to the system 100 of FIG. 1A with the exception of the structure of expandable frame 414. Frame 414 may be coupled to elongate member 406. Sheath 412 may be slidably disposed relative to elongate member 406. Frame 414 may include struts 418 and membrane 416. Electrodes 424 may be coupled to frame 414.

As can be seen by inspection of FIG. 4A in comparison with FIG. 1A, frame 414 takes the form of a double cone, in which a first section 415 is highly similar to expandable end-effector 114, but instead of terminating in an open ended structure, a second cone 417, having its base contiguous with the base of second of first section cone 415, extends distally. As can be seen, first section cone 415 is characterized by an increase in radius in the distal direction (toward the vessel wall 404), while second section cone 417 is similarly characterized by a decreasing radius in the distal direction. In some embodiments, the membrane terminates at the end of the first section cone 415, and the second cone 417 includes only the struts and not the membrane or further electrodes. An atraumatic distal tip 408 may be provided at the distal end of double cone 414.

In its collapsed state, seen in FIG. 4B, double cone 414 does not terminate with struts 418 having separate distal ends. Thus, system 400 may offer the advantage of decreased risk of tissue damage during passage through the vasculature.

It should be apparent that the medical device of the present disclosure may be used to carry out a variety of medical or non-medical procedures, including surgical and diagnostic procedures in a wide variety of bodily locations. For example, ablation of tissue associated with a variety of body organs, such as esophagus, stomach, bladder, or the urethra could be accomplished using the method discussed above. In addition, at least certain aspects of the disclosed embodiments may be combined with other aspects of the embodiments, or removed altogether, without departing from the scope of the disclosure.

Other embodiments of the present disclosure will be apparent to those skilled in the art from consideration of the specification and practice of the embodiments disclosed herein. It is intended that the specification and examples be considered as exemplary only, with a true scope and spirit of the invention being indicated by the following claims.

What is claimed is:

1. A medical device for modulating nerve activity, the medical device comprising:
a sheath;
an elongate shaft disposed in the sheath, the shaft having a distal end;
an expandable frame attached to the shaft, the frame including a plurality of struts and a membrane attached to the struts;
wherein the frame is configured to shift between an expanded configuration and a collapsed configuration;
wherein the membrane is configured to partially occlude blood flow through a blood vessel when the frame is in the expanded configuration; and
one or more electrodes coupled to the frame.

2. The medical device of claim 1, wherein the frame has a conical shape when in the expanded configuration.

3. The medical device of claim 1, wherein the membrane defines one or more pleated regions when the frame is in the expanded configuration, the pleated regions extending radially inward relative to the struts.

4. The medical device of claim 3, wherein the pleated regions are configured to increase blood flow adjacent to the electrodes.

5. The medical device of claim 1, wherein at least some of the one or more electrodes are attached to the struts.

6. The medical device of claim 1, wherein at least some of the one or more electrodes include radiofrequency electrodes.

7. The medical device of claim 1, wherein at least some of the one or more electrodes are attached to the membrane.

8. The medical device of claim 1, wherein the plurality of struts each have a distal end and wherein at least some of the one or more electrodes are disposed proximally of the distal ends of the struts.

9. The medical device of claim 1, wherein the frame has a proximal region and a distal region, the proximal region having a first shape that expands radially outward and the distal region having a second shape that converges radially inward.

10. The medical device of claim 1, wherein the frame is self-expanding.

11. The medical device of claim 10, wherein the sheath is configured to shift the frame between the expanded configuration and the collapsed configuration.

12. The medical device of claim 1, further comprising and expandable member disposed within the frame, the expandable member being configured to shift the frame between the expanded configuration and the collapsed configuration.

13. A medical device for modulation of renal nerve activity, the medical device comprising:
a sheath;
an elongate shaft slidably disposed within the sheath, the shaft having a distal region;
a self-expanding umbrella frame attached to the shaft, the frame including a plurality of struts and a membrane attached to the struts;
wherein the frame is configured to shift between a collapsed configuration when the frame is disposed within the sheath and a conical configuration when the sheath is disposed proximally of the frame;
one or more electrodes coupled to the frame;
wherein the membrane defines a plurality of pleated regions that extend radially inward relative to the struts when the frame is in the conical configuration; and
wherein the pleated regions are configured to increase blood flow adjacent to the electrodes.

14. The medical device of claim 13, wherein at least some of the one or more electrodes are attached to the struts.

15. The medical device of claim 13, wherein at least some of the one or more electrodes are attached to the membrane.

16. The medical device of claim 13, wherein at least some of the one or more electrodes include radiofrequency electrodes.

17. The medical device of claim 13, wherein the plurality of struts each have a distal end and wherein at least some of the one or more electrodes are disposed proximally of the distal ends of the struts.

18. The medical device of claim 17, wherein the plurality of struts each have a proximal end and wherein the distal ends of the struts are disposed radially outward relative to the proximal ends of the struts.

19. The medical device of claim 13, wherein the membrane is configured to partially occlude flow of blood through a blood vessel when the frame is in the conical configuration.

20. A method for modulating renal nerves, the method comprising:
providing a renal nerve modulation device, the device comprising:
a sheath,
an elongate shaft slidably disposed within the sheath, the shaft having a distal region,
a self-expanding umbrella frame attached to the shaft, the frame including a plurality of struts and a membrane attached to the struts,
wherein the frame is configured to shift between a collapsed configuration when the frame is disposed within the sheath and a conical configuration when the sheath is disposed proximally of the frame,
one or more electrodes coupled to the frame,
wherein the membrane defines a plurality of pleated regions that extend radially inward relative to the struts when the frame is in the conical configuration, and
wherein the pleated regions are configured to increase blood flow adjacent to the electrodes;
advancing the renal nerve modulation device through a blood vessel to a position within a renal artery;
proximally retracting the sheath relative to the frame;
wherein proximally retracting the sheath relative to the frame shifts the frame from the collapsed configuration to the conical configuration; and
activating at least one of the one or more electrodes.
